(19) United States
(12) Patent Application Publication
(10) Pub. No.: US 2017/0007870 A1
(43) Pub. Date: Jan. 12, 2017

(54) DEVICE FOR CHEMICAL DRIP CONTAINMENT AND NEUTRALIZATION

(71) Applicant: Chad Lavender, McKinney, TX (US)

(72) Inventor: Chad Lavender, McKinney, TX (US)

(21) Appl. No.: 14/795,769

(22) Filed: Jul. 9, 2015

(51) Int. Cl. A62D 3/30; B65D 85/00

(52) U.S. Cl. CPC A62D 3/30; B65D 85/70

(57) ABSTRACT

A chemical drip containment and neutralization device comprising a disc shaped insert impregnated with at least one neutralizing agent. The insert is adapted to be positioned within a container in such a way, so that the insert divides an inner space of the container into a liquid feed section and a liquid collecting section. The insert further comprises an air vent with a check valve and a port comprising a plug or valve to control flow of a fluid and a retaining ring placed around the circumference of the insert for holding the insert in place within the container. The insert is customizable according to container type and nature of the chemical to be neutralized.

### FIELD OF THE INVENTION

The present invention generally relates to containment and neutralization of chemicals, and more specifically relates to a self-contained insert for containers, which are capable of collecting, neutralizing and storing chemicals, the insert is customizable according to container type and nature of the chemical to be neutralized.

### BACKGROUND

Handling, storage and transport of chemicals involves multiple challenges regarding personnel safety, environmental hazard, and safe disposal to avoid contamination of land and water bodies. Especially, disposal of hazardous chemicals presents a significant environmental challenge. Chemicals in bulk quantities are used in industries like pharmaceuticals, semiconductor, water treatment, automotive, agriculture, car wash etc., where handling and disposal of chemicals forms a routine task.

Different types of containment systems have been used for chemical drip and spillage control, which include containment barrels, spill trays, containment sumps, spill berms, spill decks, rack storage systems, spillage absorbent pads, liquid proof films and the like. However the existing containment systems does not offer neutralization of hazardous or non-hazardous chemicals thus rendering them safe for disposal. For example, 55 gallon drums which are commonly used for storage and transport of hazardous chemicals offers only drip containment.

Chemicals leaking from a container or by products of chemicals resulting from industrial processes might comprise hazardous substances such as flammable substances, strong acids, strong bases, corrosives, oxidizers etc., and therefore required to be adequately treated or neutralized before disposal. A significant number of solid and liquid wastes are neutralized by high temperature incineration, which require expensive incinerator plants or transportation to such facilities. Other methods of neutralization involves addition of absorbents to the spilled or leaked chemical prior to cleaning off the spillage.

Therefore, there still exists a need for an improved device for drip containment and neutralization of chemicals, the device customizable according to different types of chemicals which are to be neutralized and also fit into varied container shapes.

### SUMMARY OF THE INVENTION

The present invention relates to a device for drip containment and neutralization of chemicals. The device comprises a disc shaped insert impregnated with at least one neutralizing agent, the insert is adapted to be positioned within a container in such a way to define a liquid feed section on top and a liquid collecting section at bottom. The insert comprises an air vent with a check valve and a port comprising a plug or valve to control flow of a fluid. The device further comprise a retaining ring placed around the circumference of the insert for holding the insert in place within the container.

In an embodiment, the insert may comprise a single layer or a plurality of layers, wherein each layer is impregnated with a different neutralizing agent. Neutralizing agents may be selected from a group consisting of bisulfite, soda ash, activated charcoal and similar agents used for neutralization of chemicals. The chemical may comprise hazardous chemical or non-hazardous chemical and the neutralizing agent is selected based on the chemical, which is to be neutralized.

In another embodiment, the insert is customizable to fit into different types of containers including buckets, drums, funnel systems, barrels and the like. The insert may further comprise a handle fastened to a top surface, which facilitates easy placement and removal of the insert.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF DRAWINGS

FIG. 1A illustrates an insert for containment and neutralization of chemicals.

FIG. 1B illustrates the insert comprising a plurality of layers.

FIG. 2A and 2B illustrates the insert placed within a bucket.

FIG. 3A illustrates the insert placed within a funnel. FIG. 3B illustrates the funnel comprising the insert, placed on a closed top drum.

FIG. 4 illustrates the insert suspended within an open top drum.

### DETAILED DESCRIPTION OF PREFERRED EMBODIMENTS

The term “insert” or “disc shaped insert” as used interchangeably herein, refers to a structure, preferably a disc shaped structure comprising a porous body or a membrane or any material which can be used for allowing passage of fluid and retain neutralization agents incorporated within the structure. The insert is configured to be positioned within a container such as bucket, drum or barrel and the like.

The term “target fluid” as used herein refers to both hazardous and non-hazardous chemicals to be contained and neutralized. The term “target fluid” may also include chemicals leaking from a container or from an apparatus or substances used in chemical warfare.

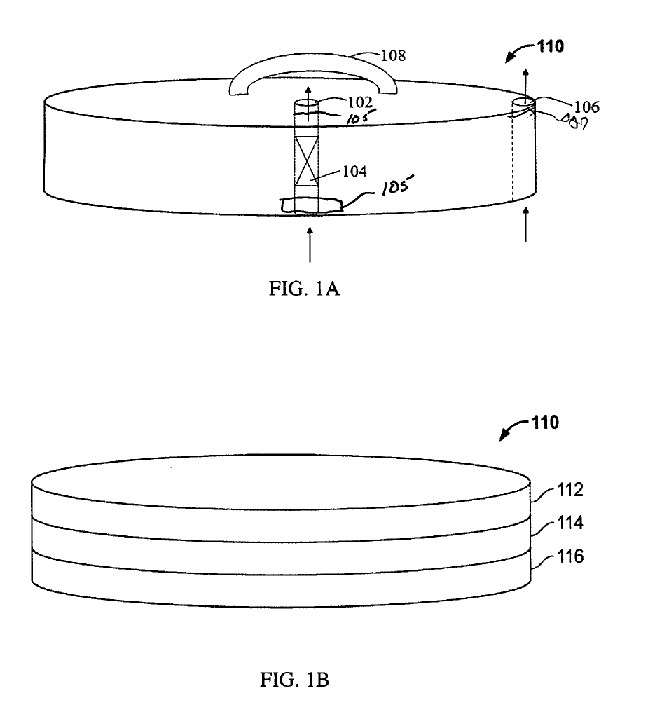

Referring to FIG. 1A, which illustrates an insert 110 for containment and neutralization of target fluid, the insert 110 comprises a disc shaped structure impregnated with at least one or more chemical agents or chemical compounds with neutralization effect. The insert 110 may comprise one or more neutralizing agents selected based on the nature of the target fluid to be neutralized. Further, the insert 110 comprises an air vent 102 which may include an O-ring 105 to provide a seal around the air vent 102 with a check valve 104 for releasing the air trapped in the area below the insert 110. The check valve 104 may include a filter 105 such as a carbon filter or other types of filters in order to filter escaping air. The check valve 104 below the insert can be a diaphragm, ball or spring type check valve. A port 106 positioned on the edge of the insert for ease of pour of the fluid. The port 106 may include an O-ring 107 to achieve a seal. The port 106 is configured to extend a cavity below the insert to a region above the insert, when placed in a container. The port 106 comprises a plug or a valve to control flow of the fluid. The insert 110 comprises a handle 108 fastened to the top surface, facilitates in easy placement and removal of the insert 110. The handle 108 may be positioned as to not interfere with the nesting/stacking of the insert 110, may be recessed with the top of the insert 110 and may be removable.

In an embodiment the insert 110 may comprise a plurality of layers 112, 114, 116 as shown in FIG. 1B, wherein each layer of the insert 110 is impregnated with a different neutralizing agent or a same neutralizing agent. A series of inserts can be added to neutralize a part or all of the captured target fluid. The insert 110 of the present invention is a self-contained device, adapted to function as a stand-alone catch and retain system.

In an exemplary embodiment, the insert 110 may comprise a single layer impregnated with soda ash as the neutralizing agent, which can be employed for neutralizing target fluids comprising strong acids such as sulfuric acid or hydrochloric acid, etc. In another example, the insert may comprise three layers comprising neutralizing agents such as bisulfite, soda ash and activated charcoal respectively, the multilayered insert can be employed for neutralizing biocides including chloride, chlorine dioxide, etc.

In an embodiment, the insert 110 may comprise porous material, sift style grading, semi-permeable membrane, cloth or any material that allows passage of fluid. The insert 110 is configured to neutralize target fluids comprising strong acids, strong bases, oxidizers, corrosives, flammable liquids, or any other fluid with extreme viscosity, pH, temperature or similar hazards. Insert 110 is intended to fit into existing commonly used containers such as 55 gallon bucket or drum, and also custom made to fit into containers of specific dimensions.

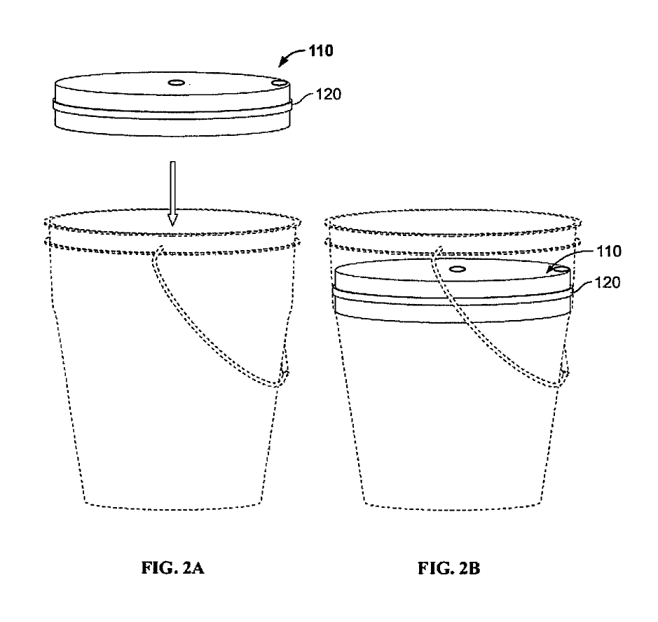

FIG. 2A and 2B, shows the insert being placed within a bucket container such as a 5 gallon tapered bucket. The insert 110 is adapted to be positioned within the container in such a way that the inner space of the container is divided into a liquid feed section on top and a liquid collecting section at bottom. The diameter of the insert 110 is slightly smaller than the inner diameter of the bucket and a retaining ring 120 is placed around the circumference of the insert 110 for holding the insert 110 in place within the container. The retainer ring 120 may comprise a rubber gasket or equivalent sealing mechanism around the circumference of the insert 110. The target fluid is gravity fed through the insert 110 where the chemicals present in the target fluid are neutralized and the neutralized target fluid is stored in the liquid collecting section at bottom of the container.

The chemical drip containment and neutralization insert 110 is a self-contained insert which can be placed within containers of different dimensions and shapes. The insert 110 can be placed in tapered or non-tapered containers such as tapered 55 gallon drums or barrels. The dimensions of the container allow the insert to wedge into the container, the insert divides the inner space of the container into a liquid feed section and liquid collection section, above and below the insert.

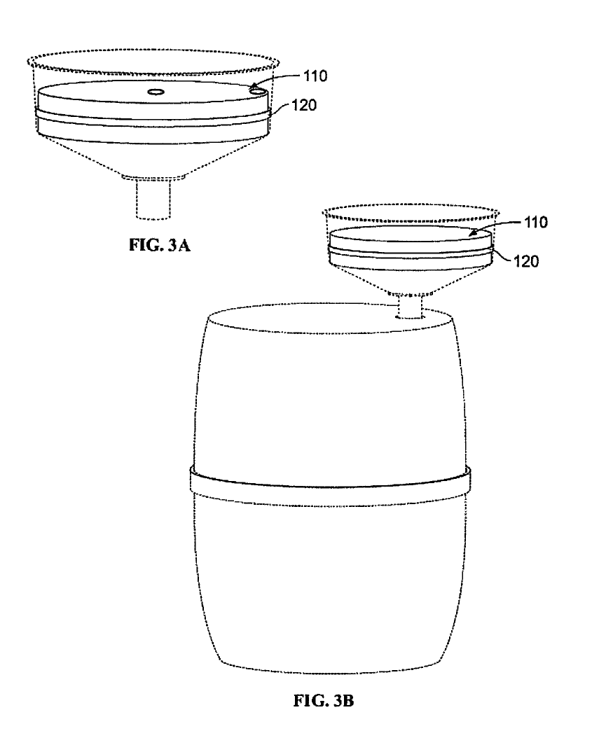

In an embodiment, the insert 110 is configured to be placed within a funnel as shown in FIG. 3A. Closed top drums or barrels comprises one or more fill ports with standard industry size ranging from 1 inch to 4 inches. The retaining ring 120 around the circumference of the insert aids in firm placement of the insert 110 within the container. The funnel comprising the insert 110 is placed in one of the ports in a closed top drum as shown in FIG. 3B. The funnel can be directly attached to the drum or attached using a standard bushing to fit into the port. In an embodiment, the insert 110 can be customized to fit into funnels with different dimensions.

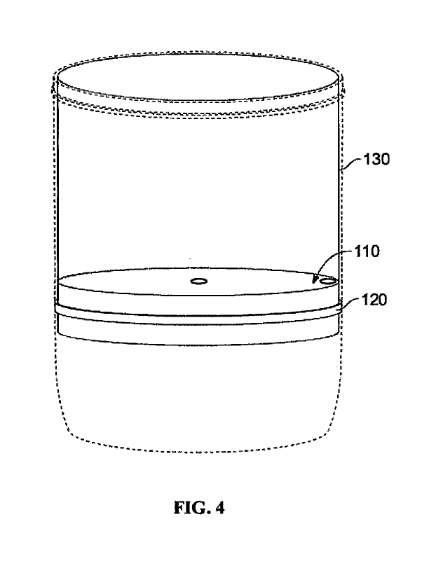

In another embodiment, the insert 110 is configured to be placed within an open top drum or barrel as shown in FIG. 4. The insert 110 comprises a side wall 130 extending upwards from the circumference of the insert. The insert 110 is suspended from the rim of the container with top edge of the side wall 130 mounted over the rim portion of the container. The side wall 130 comprise a pliable material selected based on the material compatibility with the target fluid. Side wall 130 of the insert 110 extend from neutralization layer to the top of the container and form a fluid tight cavity above the neutralization layer. The lid of the container can be secured to the top of the container with the insert placed inside or insert removed.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

1. A device for drip containment and neutralization of chemicals, the device comprising:
a disc shaped insert impregnated with one or more neutralizing agents, wherein the insert comprises an air vent with a check valve and a port comprising a plug or valve to control flow of a fluid;
a retaining ring placed around the circumference of the insert for holding the insert when placed within a container; and
a handle fastened to a top surface of the insert.

2. The device of claim 1, wherein the insert is adapted to be positioned within a container.

3. The device of claim 2, wherein the insert is positioned in such a way, so that the insert divides an inner space of the container into an upper section and a lower section.

4. The device of claim 3, wherein the upper section comprise a liquid feed section and the lower section comprises a liquid collecting section.

5. The device of claim 1, wherein the neutralizing agent is selected based on the chemical to be neutralized.

6. The device of claim 1, wherein the chemical comprises hazardous chemical or non-hazardous chemical.

7. The device of claim 1, wherein the insert comprise a plurality of layers.

8. The device of claim 7, wherein each layer is impregnated with a different neutralizing agent.

9. The device of claim 1, wherein the insert is customizable to fit into different types of containers.

10. The device of claim 1, wherein the insert is a standalone catch and retain system.
